(19) United States
(12) Patent Application Publication
(10) Pub. No.: US 2006/0231774 A1
(43) Pub. Date: Oct. 19, 2006

(54) Method and apparatus for controlled manufacturing of nanometer-scale apertures

(76) Inventors: Arnoldus Jan Storm, HT Leiden (NL); Hendrik willem Zandbergen, Katwijk ZH (NL)

Correspondence Address: DLA Piper Rudnick Gray Cary, P O Box 64807, Chicago, IL, 60664-0807, US

(21) Appl. No.: 10/547,873

(22) PCT Filed: Mar. 5, 2004
(86) PCT No.: PCT/NL04/00166

§ 371 Date: Feb. 21, 2006

(30) Foreign Application Priority Data
Mar. 5, 2003 (NL) 1022855

(51) Int. Cl. A61N 5/00

(52) U.S. Cl. 250/492.3

(57) ABSTRACT

The invention relates to a method for manufacturing nanometer-scale apertures, wherein, in an object, in a conventional manner, at least one aperture is provided with a nanometer-scale surface area, after which, by means of an electron beam, energy is supplied to at least the edge of said at least one aperture, such that the surface area of the respective aperture is adjusted, wherein the surface area of the aperture is controlled during adjustment and the supply of energy is regulated on the basis of the surface area change.

The invention relates to a method for controlled manufacturing of nanometer-scale apertures.

It is known to provide nanometer-scale apertures in objects such as membranes by means of lithographic processes. By means of these, only holes can be manufactured with a diameter which is larger than approximately 20 nm, while the reproducibility of the size of the surface areas to be obtained therewith is particularly low.

For various applications, it is very important to provide apertures with a predetermined, exact size, which, in various applications, such as for instance for chemical and DNA analysis techniques, need to be particularly small, in particular with a surface area which cannot be achieved by means of the existing lithographic techniques. In addition, for commercial applications, it needs to be possible to provide the apertures with high reproducibility.

The invention therefore contemplates providing a method by means of which apertures can be provided in objects, in particular inorganic objects, with great precision, which apertures can accurately and controlledly be manufactured with a predesired size.

The invention further contemplates providing such a method by means of which such apertures can be manufactured with high reproducibility.

The invention further contemplates providing a method for manufacturing nanometer-scale apertures by means of which such apertures can be provided in relatively thin membranes.

The invention further contemplates providing such a method by means of which no foreign materials are added to the object in which the or each aperture has been provided.

The invention further contemplates providing an apparatus, at least assembly by means of which nanometer-scale apertures can be manufactured, at least adjusted in size and/or shape, with high accuracy.

At least a number of these and other objects are achieved according to the invention with a method according to claim 1.

With a method according to the invention, one or more apertures can be provided in an object, by means of conventional techniques such as lithographic techniques, of which the size and, optionally, the shape can then be adjusted. The adjustment of the size and/or shape can continuously be controlled, on the basis whereof the energy supply for this adjustment can simply and accurately be regulated. This may, for instance, be: done by regulation of the intensity and/or the spot size, at least the beam size of the electron beam used for this purpose.

Because the size of the aperture, at least the change thereof, is recorded during the supply of the energy, real-time regulation is possible, so that the size can particularly accurately be adjusted. This means that a particularly high yield can be obtained, with a particularly high reproducibility. In addition, such a method can be carried out relatively simply and relatively inexpensively.

By using an electron beam for the supply of energy, the aperture can be adjusted without foreign materials needing to be added.

It has surprisingly been found that, with a method according to the invention, apertures can both be reduced and can be enlarged, in particular depending on the initial size of the aperture. Without wishing to be bound to any theory, this seems to be the result of the free energy and the surface area size. Apertures with a diameter of the order of magnitude larger than the thickness of the object in which the aperture has been provided will increase in size, while smaller apertures will reduce upon supply of energy according to the invention. This effect seems to occur at least in highly viscous materials such as glasslike materials, for instance Si-based materials. The radius at which the transition point occurs between apertures which will grow and apertures which will shrink is referred to as critical radius and partly depends on the material used and in particular the geometry of the initial aperture. In this application, initial aperture is at least understood to mean an aperture provided by means of conventional techniques, and apertures which have not yet been controlledly brought to the accurate, desired size by means of a method according to the invention.

Because, in a method according to the invention, the rate of growth or decrease in the size of the aperture can accurately be controlled on the basis of the changes observed in real time, by regulation of the electron beam, this change can be stopped at any desired moment. Consequently, a high accuracy can be achieved.

In a method according to the invention, preferably, a membrane-shaped object is used, provided with a core with thereon at least one layer of material with a highly viscous, glasslike behavior, preferably Si-based material such as SiO₂. This layer is preferably provided on two opposite sides of the core, as well as on the surfaces of the aperture, such that the core is coated by this layer at least near this aperture. Then, to this layer, energy is supplied by means of the electron beam, In such a manner, relatively thin, preferably inorganic membranes can simply be manufactured with small nanometer-scale apertures with an accurately determined surface area, while an electrically conductive layer may be provided close under this layer. This may, for instance, be advantageous for being able to regulate a surface tension, so that, for instance, the possibility is offered to externally control the interactions between the wall of the respective at least one aperture and negatively charged DNA molecules.

In this specification, aperture should at least be understood to mean a passage in an object, in particular a membrane, open to two opposite sides of this object. The surface area thereof, at least the size of this surface area or the diameter thereof, should at least be understood to mean the smallest surface area, at least the smallest diameter of the aperture, viewed in axial direction. As a measure for this, for instance the surface area, at least the diameter of a beam of parallel rays which can fall unhindered through the aperture, parallel to the axial direction, can be taken. In this application, nanometer-scale should at least be understood to mean linear dimensions between 0 and approximately 1,000 to 10,000 nanometers (nm).

For controlling the size of the aperture, at least the diameter thereof and/or changes thereof, according to the invention, use is particularly made of visual means such as a CCD screen or a fluorescent screen. For the determination of the surface area size and/or the diameter of the aperture, preferably, use is made of known polygon tracing techniques, in which the circumference of the aperture is enclosed by this polygon, then the enclosed surface area thereof is calculated and this is translated to a diameter of a circle with the same surface area. Where, in this application, further, the term diameter is used, referring to the aperture, this diameter will be intended, unless clearly indicated otherwise.

Techniques for manufacturing the initial apertures are known from practice and are for instance described by Gribov et al “New fabrication process for metallic point contacts”; Microelectronic Engineering; 35, 317-320 (1997), inserted herein by reference.

In a method according to the invention, preferably, use is made of a known electron microscope, in which the electron beam is regulated on the basis of the observed size, at least changes therein, of the aperture. This has the important advantage that such microscopes are simply and generally available, while they are relatively simple to operate and relatively inexpensive in use.

Incidentally, the initial aperture may also be provided by means of an electron beam, with relatively high energy level, after which the size of the aperture may then be adjusted according to the above-described manner by means of lower-energy electron beams.

In an advantageous embodiment of the invention, the or each aperture is provided in a glasslike material which becomes softer as a result of supply of energy according to the invention. Herein, glasslike material should at least be understood to mean material with, near room temperature, an at least largely amorphous structure which behaves like a supercooled fluid with a particularly high viscosity, such that it behaves like a solid in the short and long term. Such materials become softer upon supply of heat, allowing controlled local deformations as a result of local heating. By using an electron beam for the supply of the energy, on the one hand, the heating can be obtained and, on the other hand, each change can directly be observed in real time, enabling regulation on the basis thereof.

On a macroscopic scale, the dynamics of these glasslike materials is determined by, on the one hand; the surface tension and, on the other hand, gravity. On the nanometer scale, the surface tension will overcome the influence of gravity and be the most important factor for change.

Preferably, the rate at which the apertures are adjusted in size is regulated such that the increase or decrease of the diameter of the aperture is, for instance, less than approximately 1 nm after removing the electron beam. This rate can simply be regulated by adjusting the energy level of the electron beam and can, for instance, be set at a growth or decrease of some tenths of nanometers per minute, in particular when the desired size is approached.

The invention further relates to the use of an electron microscope for controlledly and accurately providing nanometer-scale apertures in objects such as membranes. It has surprisingly been found that, while, usually, preparations are not physically influenced by an electron microscope, by means of an electron microscope, particularly small apertures can particularly accurately be manufactured, at least nanometer-scale apertures can be accurately adjusted to a specific, desired size. Use of an electron microscope for this purpose is particularly advantageous because of the availability and the costs of use thereof. In addition, it can be used without addition of external materials.

The invention further relates to an assembly for providing and/or adjusting nanometer-scale apertures in objects such as membranes, characterized by the measures according to claim 17.

Such an assembly, which is particularly suitable for use of the above-described methods, is simple in construction and use, is relatively robust and is easy to assemble.

The invention further relates to an object, in particular a membrane, characterized by the measures according to claim 21, 22 or 24.

Such objects offer the advantage that they have a high accuracy, in particular with regard to an aperture or apertures provided therein, while they can be manufactured relatively inexpensively and with high reproducibility.

In the further subclaims, further advantageous embodiments of the invention are described.

In order to explain the invention, embodiments of a method, use, assembly and object according to the invention will be further elucidated with reference to the drawing, in which:

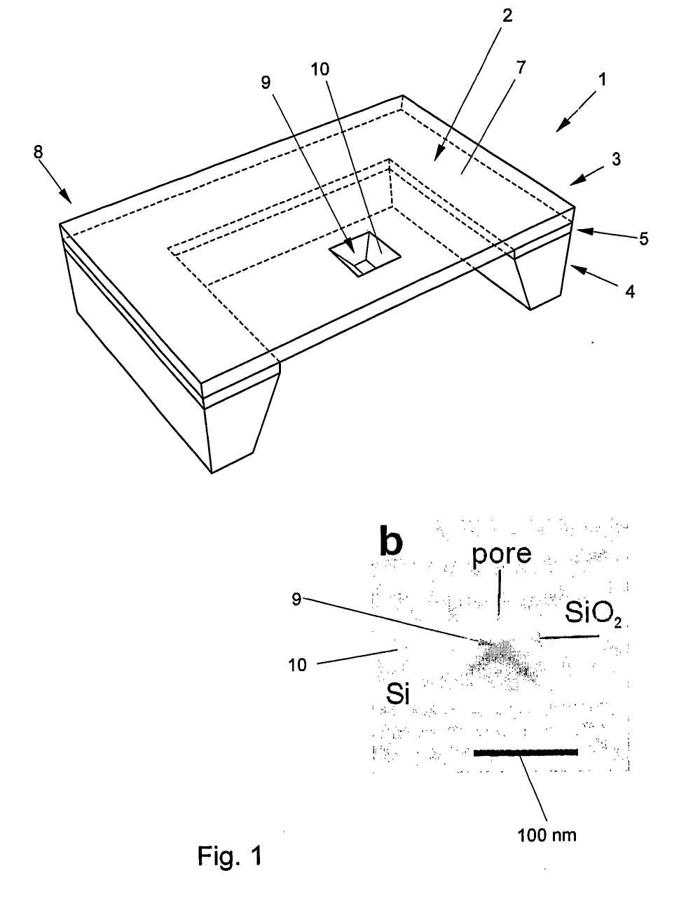

FIG. 1 diagrammatically shows, in perspective view, an object, in particular a free-standing membrane according to the invention, with nanometer-scale aperture, prior to adjustment of the dimension thereof, and the aperture in top plan view;

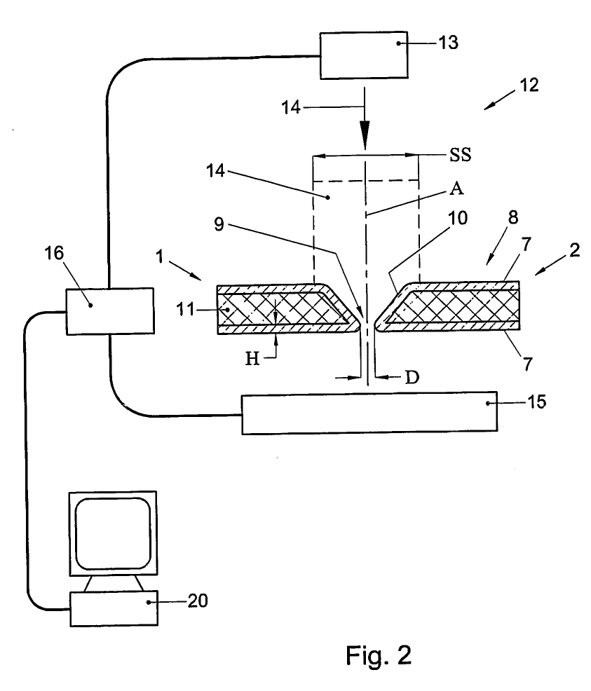

FIG. 2 diagrammatically shows, in sectional side elevational view, an assembly according to the invention, during supply of energy by means of an electron beam;

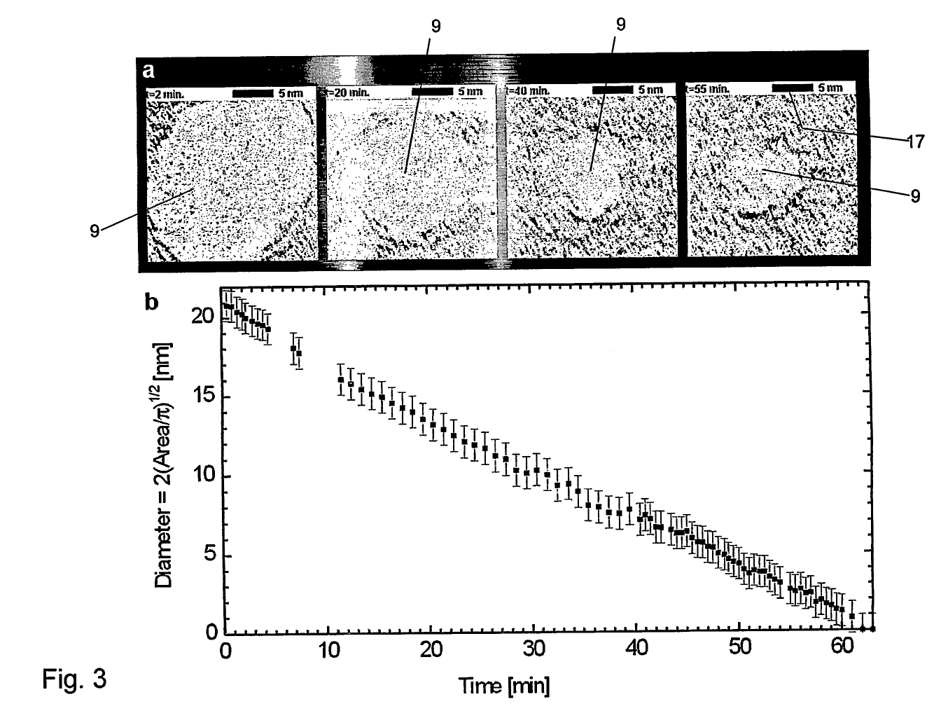

FIG. 3 shows four images of an aperture as shown in FIGS. 1 and 2, during supply of energy, which show the decrease in size, and a diagram in which the diameter change of the aperture has been plotted against time;

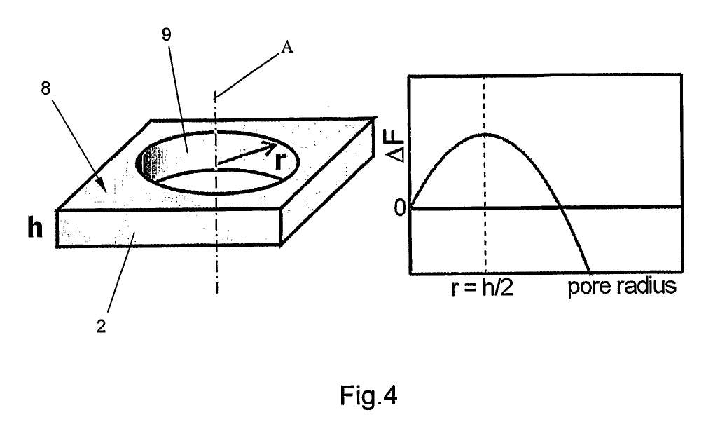

FIG. 4 shows, graphically plotted, the free energy plotted against the radius of the aperture;

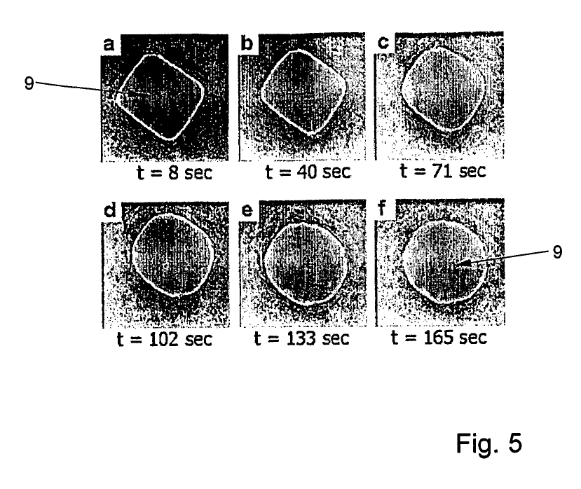

FIG. 5 shows, in six recordings, the change in shape of an aperture;

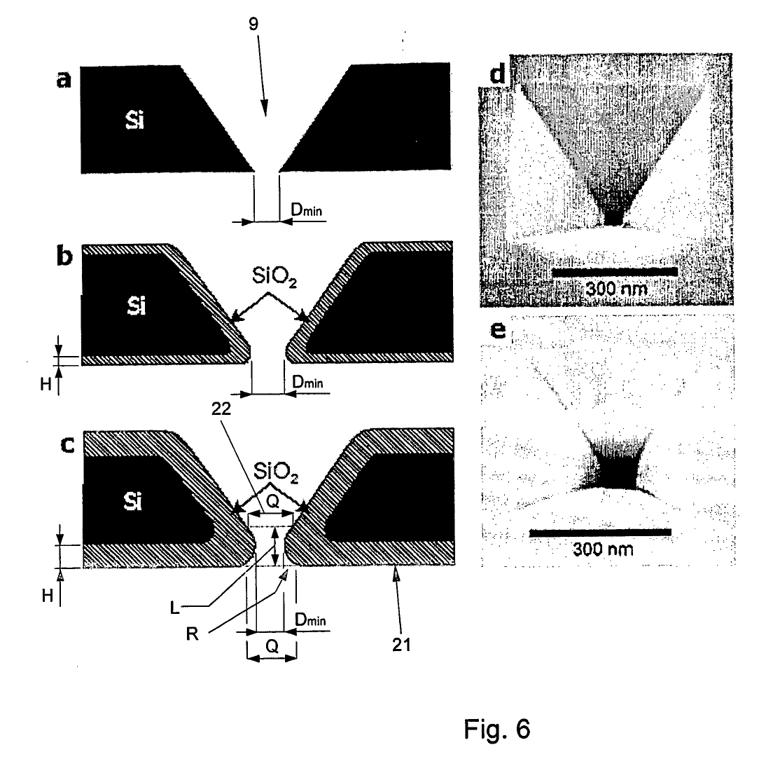

FIGS. 6a-c show, in sectional side elevational views, the change in shape of an aperture;

FIGS. 6e and 6f show the initial aperture according to FIG. 6a and the final aperture according to FIG. 6c, respectively;

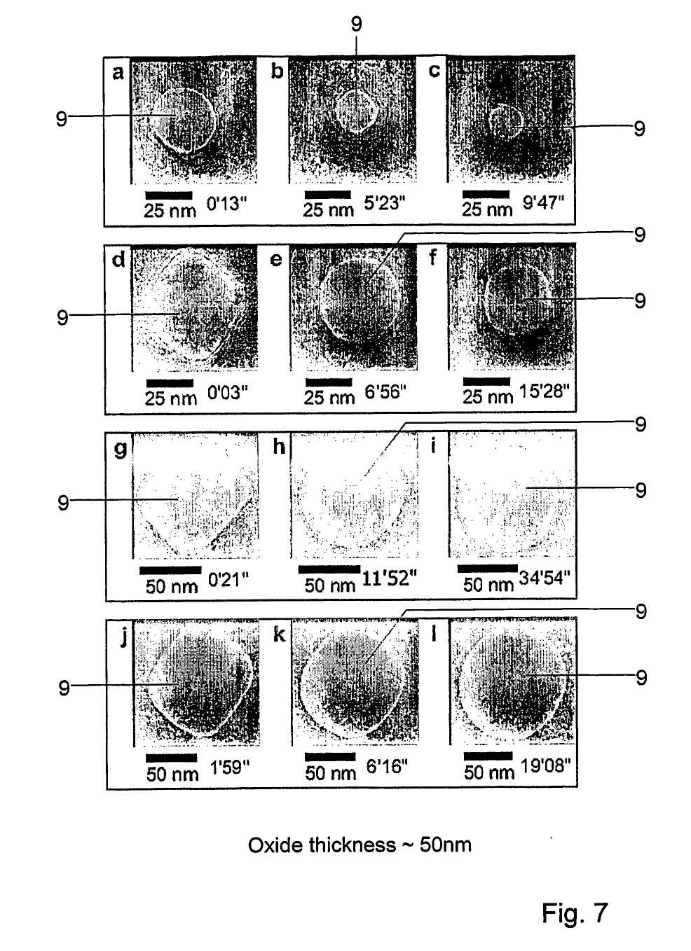

FIGS. 7a-c, d-f, g-i, j-l show changes of an aperture as a result of a method according to the invention, with different initial aperture sizes, with a thickness of the material of approximately 50 nm;

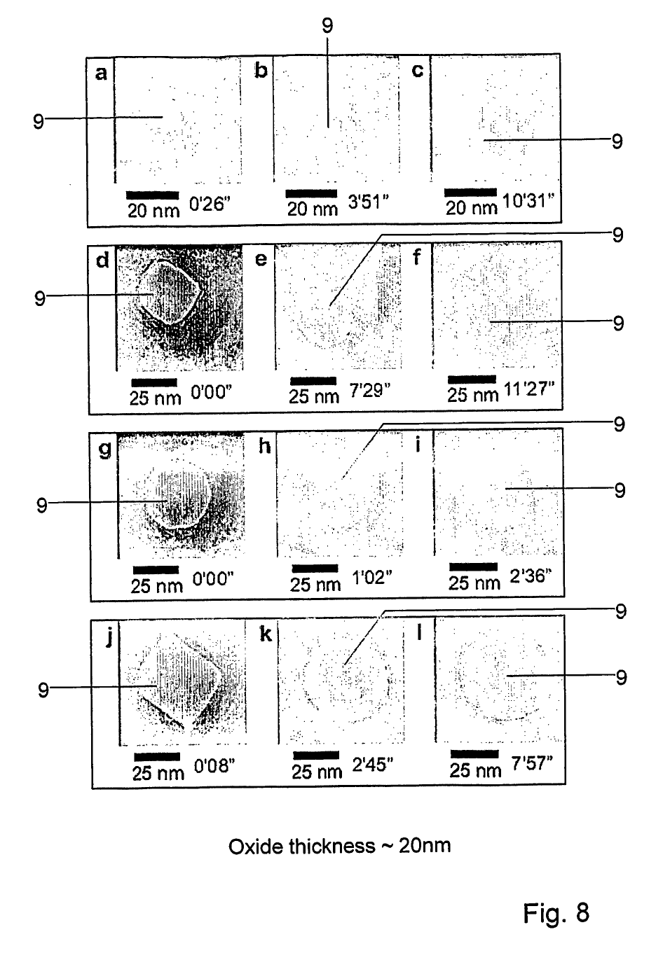

FIGS. 8a-c, d-f, g-i, j-l show changes of an aperture as a result of a method according to the invention, with different initial aperture sizes, with a thickness of the material of approximately 20 nm; and

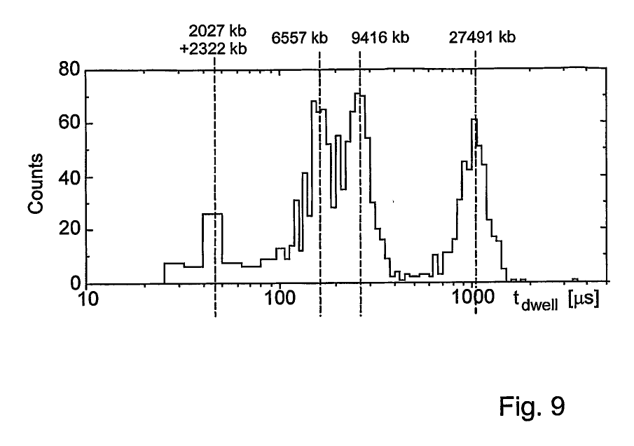

FIG. 9 shows a histogram of a measurement of molecule lengths in a mixture of molecules.

In this description, same or corresponding parts have same or corresponding reference numerals. In this description, as an object, a free-standing membrane is shown. However, of course, in a similar manner, apertures may be provided in other objects, in particular in relatively thin products. As an exemplary embodiment for providing initial apertures, lithography is described. However, various other existing, known techniques for making nanometer-scale apertures may be used, for instance by use of a high-energy electron beam.

FIG. 1 diagrammatically shows, in perspective view, a part of an object 1 according to the invention, prior to adjustment of an aperture. In the embodiment shown, this is a free-standing membrane formed from a Silicon On Insulator (SOI) wafer. For the manufacture thereof, in the exemplary embodiment shown, as starting material, an SOI is taken with a top single-crystal silicon layer 3 of 340 nm with a crystal orientation <100> carried by a Si wafer 4 of approximately 525 nm and a layer 5 of SiO₂ provided between the wafer 4 and the top layer 3. By use of micromachine technique, therefrom, free-standing silicon membranes of approximately 70 by 70 nm were manufactured. This is a membrane 2 formed by the top layer 3 of 340 nm, carried by an edge 6 of the original wafer 4 and the intermediate layer 5 of the SOI. The top layer 3 is then, by oxidation, in particular thermal oxidation, provided with an approximately 40 nm-thick coating layer 7 of SiO₂, preferably on both sides thereof, as FIG. 2 clearly shows, but in any case on the top side 8, at the location where an aperture 9 needs to be provided.

By using e-beam lithography and reactive ion etching, substantially rectangular, in particular approximately square apertures are provided in the top coating layer 7, with sides of approximately 200 nm to 500 nm, after which, thereupon, slightly pyramid-shaped cavities 10 are etched by means of wet KOH etching. After stripping the oxide in a buffered HF, the membranes 2 are thermally oxidized with a coating layer, again of approximately 40 nm, which also extends along the inner surface of the pyramid-shaped cavity 10. The (initial) aperture 9 as such is also rectangular and forms the top of the pyramid (directed downwards). This has a surface with sides of approximately 20 nm. The core 11 of the silicon membrane 2 is coated towards the outside, at least near the aperture 9.

Such a technique is generally known per se and is described in the above-mentioned article of Gribov et al, which is understood to be inserted herein by reference.

As shown in FIG. 2, the object 1 is received in an assembly 12 according to the invention, in the exemplary embodiment shown in a specimen holder (not shown) of an electron microscope 13. Here, the membrane 2 is positioned such that the aperture 9, at least the cavity 10, is placed in the electron beam 14 of the microscope 13 with the wide side facing up, with the longitudinal direction of the beam 14 being approximately equal to the axial direction A of the aperture 9, at least being approximately perpendicular to the top side 8 of the membrane 2. In the exemplary embodiment shown and described, a HR-TEM electron beam 14 is used of approximately 300 kV, with a spot size of approximately 200 nm to 500 nm, at least approximately similar to the dimensions of the wide side of the cavity 10.

Energy level and spot size may, of course, be adjusted as desired, as will also be described hereinafter and may be calculated or be experimentally determined depending on, for instance, the initial dimensions of the cavity 10 and the aperture 9, the material of the membrane, the desired rate of change of shape and the like.

On the side located opposite the top side 8 of the membrane, in FIG. 2 thus under the membrane 2, a visual recorder 15 such as a CCD camera or a fluorescent screen is arranged, by means of which, continuously, an image can be obtained of the dimensions and the shape of the aperture 9. This recorder 15 is coupled to a regulating device 16 in which at least an algorithm is included to calculate the surface area of the aperture 9 from the image by means of polygon tracing, and to determine the diameter of a circle with similar surface area therefrom. As a result of the energy supplied by the electron beam 14, the material will become slightly softer and, as a result of gravity and particularly surface tension, will move, at an atomic level, so that the dimension and, optionally, the shape changes to a condition with a lower free energy (F), as will be described in more detail.

By means of the regulating device 16 with computer 20 and the diameter Dₜ of the aperture calculated by means of the algorithm, it can be determined whether a desired size D_{w} of the aperture 9 has been reached, while, thereby, on the basis of the diameter Dₜ, the electron beam 14 can continuously be regulated in real time in, for instance, energy level and/or spot size. In this manner, the rate of change can be regulated and the beam 14 can be removed if the desired diameter D_{w} has been reached.

FIG. 3 shows, in four images, starting from an aperture 9 with an initial diameter, at least approximately, of about 21 nm, the reduction of the diameter D in time. In each Figure, the time t after the start of supply of energy is indicated, and a black bar 17 corresponding to a length of 5 nm. It is clear that the surface area of the aperture 9 decreases, in the example shown from the initial size to a size with a diameter of approximately 3 nm in approximately 55 minutes, while the aperture 9 is virtually completely closed after approximately 1 hour. The diagram shown below the four images shows that the rate of change of the diameter was approximately 0.3 nm per minute, so that the change can be stopped within approximately 1 nm. Of course, this rate can be increased or decreased by adjusting particularly the energy level.

It is clear that, at higher energy levels, it will be possible to obtain sharper images, in particular because less diffraction and scattering will occur near the edges of the aperture, but that, here, higher rates in change and, consequently, a more difficult controllability of the decrease in the diameter and the final diameter D_{w} are obtained. It can be advantageous to initially choose the energy level so as to be relatively high and to lower it when the desired diameter D_{w} is approached. Thus, the material returns to the “frozen” original state.

FIG. 4 shows the free energy F, at least the change therein dF, plotted against the radius of the aperture 9. The free energy can be determined by the formula dF=σdA, in which dA=2π(rh−r²) is the change in surface area from an intact surface to a surface with a cylindrical aperture 9 with radius r and a thickness h, and σ is the surface tension. The graph shows that the free energy is maximal for a radius of the aperture 9 which is approximately equal to half the thickness h of the object in which the aperture 9 has been provided, such as the membrane 2. Apertures with a radius r smaller than approximately h/2 can lower their free energy by reducing, larger holes by enlarging. In the exemplary embodiment shown, it is therefore true that, as a result of supply of energy by the electron beam 14, the radius of the aperture 9 will decrease. With holes larger than a radius of approximately 80 to 100 nm, in the exemplary embodiment shown of a membrane 2, the diameter will increase upon supply of energy. The radius r_{c} for which the free energy is maximal can be referred to as the critical radius. For manufacturing particularly small apertures (with a radius between for instance 0 and some tens of nanometers), it is therefore only important that the initial diameter remains below the critical radius.

With a membrane 2 according to the invention, below the coating layer 7, an electrically conductive layer is obtained, so that voltage changes in the aperture 9 can be recorded or an electrical voltage can be applied to the wall of the aperture 9. This is particularly advantageous for, for instance, research into DNA, more in particular translocation studies such as for instance described by Li et al, Ion-Beam Sculpting at Nanometer length Scales, Nature 412, 166-169 (2001), inserted herein by reference, when Si₃N₄ and nano-apertures are used. An object 1, in particular a membrane 2 according to the invention, is particularly suitable for such research, particularly because apertures 9 can be obtained with an accurate diameter of for instance one or a few nanometer(s). However, with a method and assembly according to the invention, nanometer-scale apertures can also be adjusted for various other uses, for instance for microelectronics, micromechanics and similar uses.

With a method according to the invention, no foreign materials are added, so that a pure membrane is preserved.

In each of the exemplary embodiments shown, a glasslike material such as SiO₂ has been taken as a starting material. It will be clear, however, that apertures in other grasslike materials can be adjusted in a similar manner, in which energy levels and the like can be experimentally determined. A method according to the invention further offers the advantage that the supply of energy is very local, so that structures at a distance from the aperture are not influenced by this. Consequently, microelectronic structures can be integrated on a chip, as well as nanometer-scale apertures with accurate dimensions. The initial apertures do not necessarily need to have a round shape but may also, as shown, have a rectangular, square, oval or odd shape. In an assembly according to the invention as shown in FIG. 2, a commercially available electron microscope can be used, with integrated visual recorder 15. However, also, an assembly may be assembled especially for the invention. Apertures 9 may also be provided and adjusted in other objects than free-standing membranes. The techniques to provide the initial apertures may be chosen relatively freely and do not need to be particularly accurate. Of course, also, multiple apertures may be provided.

As FIG. 5 shows, by means of a method according to the invention, an initially rectangular aperture (FIG. 5a) can be made round (FIG. 6c), which may be advantageous for uses in which angular apertures are less advantageous, for instance for selectively passing round, spherical and/or cylindrical particles, specific molecules and the like.

FIG. 6 shows, in cross section, a membrane according to the invention, in three steps (a-c), during the manufacture of a desired aperture 9. This is similar to the object 1 as shown and described in FIGS. 1 and 2. FIG. 6a shows the initial aperture 9, FIG. 6c the final aperture 9. FIG. 6b shows an intermediate stage. FIG. 6d shows an electron microscope recording of the initial aperture 9 according to FIG. 6a, FIG. 6e of the final aperture according to FIG. 6c. It is clear from FIGS. 6a-c that the minimal cross section Dₘᵢₙ of the aperture 9 changes during the method according to the invention. When the thickness (H) of the coating layer 7 increases, the curvature radius R of the edges of the aperture 9 becomes increasingly larger, so that the length L of the aperture increases. When the length L increases, the size of the initial aperture 9 will increase, while, with a method according to the invention, the aperture 9 will still shrink. This is shown in FIGS. 7 and 8. The length L of the aperture 9 is approximately equal to the distance between the surface opposite the top side 8, further also to be referred to as the bottom side 21, and the cross section 22 of the aperture 9, which has a diameter Q which is approximately equal to the diameter Q of the aperture 9 in the bottom side 21.

FIG. 7 shows, in four series a-c, d-f, g-i and j-l, recordings of apertures 9 in an apparatus 1 with an oxide layer with a thickness of approximately 50 nm. With the aperture 9 with an initial diameter of approximately 40 nm, the aperture 9 becomes smaller and remains round (FIGS. 7a-c). With an initial diameter of approximately 55 nm, the aperture also becomes slightly smaller and becomes rounder (FIGS. 7d-f). With an initial diameter of approximately 80 nm, the aperture stays approximately equally large, but becomes clearly rounder (Figs. g-i), while, with an aperture of approximately 100 nm, the size increases relatively slowly and the aperture also becomes rounder (Figs. j-l). In each FIG. 7a-l, the time from a point in time 0 is shown as the moment that the respective recording was made.

FIG. 8 shows images similar to those according to FIG. 7, in which, however, an oxide layer 7 of approximately 20 nm has been taken as starting material. So, the length is smaller than the length of FIG. 7. As is shown, with an aperture of approximately 15 nm, the aperture 9 will shrink (FIGS. 8a-c), while an aperture of approximately 30 nm will stay approximately the same as regards surface area and will become rounder (FIGS. 8d-f). With an aperture 9 of approximately 35 nm, a slow increase of the surface area will clearly occur (FIGS. 8g-i), while an aperture of approximately 40 nm will grow fast and will, in addition, become rounder (FIGS. 8j-l). It is clear that the length L of the aperture 9, at least the thickness of the oxide layer, influences the change in shape of the aperture.

FIG. 9 shows a histogram of a test in which molecules of different lengths have been pulled through an aperture 9 by means of a magnetic field. As molecules, DNA molecules have been used for this. An electric field was applied by means of a potential difference between the two sides of the aperture 9. A mixture of molecules with different lengths was brought above the top side 8 and, by means of the electric field, molecules were pulled through the aperture 9, one by one. The time (t) needed for passing the aperture 9 by each individual molecule can be measured by change in the field. This is because, during the time when a molecule is received in the aperture, the aperture is at least partly closed off. In FIG. 9, it is indicated for the molecules how many molecules took a certain time to pass the aperture 9. Here, the position on the horizontal axis indicates a measure for the duration that the aperture was blocked by a respective molecule, and, accordingly, for the length of the molecule, and the vertical axis for the frequency at which the respective molecule was present in the solution. A longer duration (t) resulted in a position more to the right on the horizontal axis. For instance, a molecule of 27491 kb needed approximately 1,000 μs to pass the aperture. Above the histogram, for a number of peaks, the respective molecule length in kb is indicated. The horizontal axis has a logarithmic scale.

Herein, unless explicitly stated otherwise, “approximately”, “substantially” or similar relative terms referring to a value or quantity are at least understood to also comprise a deviation of at least 20%, in particular at least 10% and more in particular at least 5% of the respective value or quantity.

These and many similar variations are considered to fall within the scope of the invention set forth in the claims.

1. A method for manufacturing nanometer-scale apertures, wherein, in an object, in a conventional manner, at least one aperture is provided with a nanometer-scale surface area, after which, by means of an electron beam, energy is supplied to at least the edge of said at least one aperture, such that the surface area of the respective aperture is adjusted, wherein the surface area of the aperture is controlled during adjustment and the supply of energy is regulated on the basis of the surface area change.

2. A method according to claim 1, wherein the surface area of the at least one aperture is reduced by supply of said energy.

3. A method according to claim 1 or 2, wherein the at least one aperture is provided in a glasslike material which becomes softer as a result of local supply of energy.

4. A method according to any one of the preceding claims, wherein the shape and the surface area of the initially provided at least one aperture are chosen such that, upon supply of said energy, under the influence of gravity and surface tension, the respective aperture gets and/or preserves a substantially circular surface and that, at the same time or subsequently, the diameter thereof reduces.

5. A method according to claim 4, wherein the at least one aperture is provided in a plate-shaped object with a thickness which approximately corresponds to or is larger than the diameter of the aperture.

6. A method according to any one of the preceding claims, wherein the at least one aperture is provided in a SiO₂ layer or the edges of the aperture are coated with a layer of SiO₂, prior to supply of the energy for adjustment of the aperture.

7. A method according to any one of the preceding claims, wherein, for the supply of energy, use is made of an electron microscope.

8. A method according to any one of the preceding claims, wherein, for the control of the surface area size, at least the diameter or radius of the at least one aperture, use is made of an image recording device such as a CCD screen or a fluorescent screen and image analysis software.

9. A method according to any one of the preceding claims, wherein the at least one aperture is provided in a free-standing silicon membrane, preferably in a microchip, wherein micro manufacturing techniques are used for the formation of a membrane, in particular with a top single-crystal silicon layer, which membrane is provided with a SiO₂ layer, preferably on two opposite surfaces, after which the at least one aperture is provided with standard technique, in particular etching technique such as e-beam lithography and reactive ion etching, wherein, thereupon, the edges of the at least one aperture are provided with a SiO₂ layer, after which the said energy is supplied for adjustment of the dimension of the respective aperture.

10. A method according to claim 9, wherein said membrane is manufactured with a thickness between 10 and 1,000 nm, in particular between 100 and 800 nm, more in particular between 250 and 650 nm, and preferably between 300 and 400 nm.

11. A method according to claim 9 or 10, wherein a SiO₂ layer is provided with a thickness which is considerably smaller than the thickness of the membrane, for instance 0.05 and 0.5 times the thickness of the membrane or less.

12. A method according to any one of the preceding claims, wherein the at least one aperture initially has a surface area approximately corresponding to that of a circle with a diameter of less than approximately 100 nm, more in particular less than approximately 80 nm.

13. A method according to any one of the preceding claims, wherein the rate of adjustment of the at least one aperture is regulated by regulation of the amount of energy supplied per time unit.

14. Use of an electron microscope for controlledly adjusting the surface area of a nanometer-scale aperture in an object, in particular a membrane.

15. Use of an electron microscope according to claim 14, wherein the surface area of said at least one aperture is reduced.

16. Use of an electron microscope according to claim 14, wherein the surface area of said at least one aperture is enlarged.

17. An assembly of a device for directedly emitting an electron beam, a device for observing an aperture in an object, at least changes in this aperture, and a regulating device for controlling the device for emitting the electron beam on the basis of signals coming from the device for observing the aperture, at least changes therein.

18. An assembly according to claim 17, wherein at least as device for emitting the electron beam, an electron microscope is provided.

19. An assembly according to claim 17 or 18, wherein the regulating device is designed for regulating at least the intensity and/or the spot size of the electron beam.

20. An assembly according to any one of claims 17-19, wherein the regulating device is provided with an algorithm for calculating the surface area of an aperture by polygon tracing of the circumference of the respective aperture and determining, on the basis thereof, the diameter of the aperture, assuming that it is circular with a surface area corresponding to the surface area determined by said polygon tracing.

21. An object, provided with at least one nanometer-scale aperture, manufactured with a method according to any one of claims 1-13, via a use of an electron microscope according to any one of claims 14-16 or with an assembly according to any one of claims 17-20.

22. An object provided with at least one nanometer-scale aperture, preferably according to claim 21, provided with at least one membrane-shaped part and therein at least one aperture, which aperture is provided with edges from SiO₂.

23. An object according to claim 22, wherein said membrane-shaped part is provided with a carrier coated with a SiO₂ layer on at least one and preferably two opposite sides, wherein the or each aperture extends between said sides, and the edges thereof are coated with a SiO₂ layer such that said core is, at least near said aperture, coated towards the environment by said SiO₂ layer.

24. An object for DNA studies, in particular DNA translocation studies, provided with at least one aperture manufactured with a method according to any one of claims 1-13, via a use of an electron microscope according to any one of claims 14-16 or with an assembly according to any one of claims 17-20.
